(19) United States
(12) Patent Application Publication
(10) Pub. No.: US 2018/0185492 A1
(43) Pub. Date: Jul. 5, 2018

(54) MICRO-NANO MEDICAL ADSORBENT RESIN POWDER MATERIAL

(71) Applicant: Jie YU, Beijing (CN)

(72) Inventor: Jie YU, Beijing (CN)

(21) Appl. No.: 15/736,792

(22) PCT Filed: Jun. 22, 2016
(86) PCT No.: PCT/CN2016/086677

§ 371 Date: Dec. 15, 2017

(30) Foreign Application Priority Data
Jul. 9, 2015 (CN) 201510399777.4
Jul. 25, 2015 (CN) 201510441375.6
Aug. 19, 2015 (CN) 201510510251.9
Aug. 19, 2015 (CN) 201510510311.7
Jan. 16, 2016 (CN) 201610028904.4
Jan. 25, 2016 (CN) 201610047350.2
Feb. 27, 2016 (CN) 201610109578.X

(51) Int. Cl. A61K 47/32; A61K 9/14; C08F 212/36; C08F 218/08; C08F 212/08

(52) U.S. Cl. CPC A61K 47/32; A61K 9/14; C08F 212/08; C08F 218/08; C08F 212/36

(57) ABSTRACT

A micro-nano medical adsorbent resin powder material is a high-molecular polymer including carbon ions, hydrogen ions, and oxygen ions. The powder material includes a molecular structure includes a benzene ring and a hydroxyl group, and the polymer includes at least two or more molecules; an overall particle size is in a range of 0.01-50 micrometer (um), preferably 0.01-1.5 micrometer (10-1500 nm).

### TECHNICAL FIELD

Medical materials

### Background

In the past, the material shape of the macroporous adsorption resin for medical use was in a regular granular form, and the macroporous adsorption resin was used as an adsorbent filled in the hemoper fusion device in the blood purification therapy. The range of the particle size is 80 mesh (178 μm). No medical resin with micro-nano particle size has been reported in the literature.

### SUMMARY OF THE INVENTION

The objectives of the present invention are to provide a technical solution of a medical absorbent resin material having a micro-nano particle size and a technical solution of using the material.

The contents of the present invention are as follows: A micro-nano medical adsorbent resin powder material, characterized in that: it is a high-molecular polymer including carbon ions, hydrogen ions, and oxygen ions; the molecular structure includes a benzene ring and a hydroxyl group, and the polymer includes at least two or more molecules; the overall particle size is in a range of 0.01-50 micrometer (um), preferably 0.01-1.5 micrometer (10-1500 nm).

### DETAILED DESCRIPTION OF THE INVENTION

Embodiments of the present invention:

### Embodiment 1

The polymer is a polymer including vinyl acetate and divinyl-benzene.

### Embodiment 2

The polymer is a polymer including styrene and divinyl-benzene.

### Embodiment 3

A carbohydrate chains is bound to a binding site of a carbon skeleton and/or a branched-chain of the polymer to form a glycosylated polymer.

### Embodiment 4

Transition-metal ions are embedded in the carbon skeleton of the polymer; and the metal element is iron element.

Embodiments of the use of the micro-nano medical absorbent resin powder material of the present invention:

### Embodiment 5

The powder material is used as a coating agent of skin, body tissue, living organ or a surface of living organism.

### Embodiment 6

The powder material is used as a vector carrying a drug or a life-intervention substance, including a stem cell.

### Embodiment 7

The powder material is used as a medical molding material, including a bulking agent for a hollow fiber, a silicone blood transfusion tube, a silicone infusion tube, a dialysis membrane, or a medical coating, or a shaping material integrated with a surface of the hollow fiber, the silicone blood transfusion tube, the silicone infusion tube, the dialysis membrane, or the medical coating.

### Embodiment 8

The powder material is used as a solute, and an injection solution or a physiological saline or ethanol having different purities, including fruit wine is used as a solvent to prepare a solution.

### Embodiment 9

The powder material is added into a shaping material to make a paste, or a pill, or a grease agent.

In the above-mentioned embodiments 7 to 9, the powder material of the present invention is added to a molding material or a shaping material or a solution, in which the maximum proportion of the powder material is limited by the maximum powder material that can be added, and the minimum proportion of the powder material is not limited. Under the conditions, without affecting the use of molding material or shaping material or solution, the amount of added powder material determines the effect.

The micro-nano effects and quantum effects of the material of the present invention are shown as follows: In the micro-nano material interface microenvironment, micro-nano particles of the adsorption resin can penetrate the skin, muscle tissue and cell membrane, and can have collisions with affinitive adsorption materials actively driven by equilibrium potential, so that the adsorption materials, including active cells, intracellular genetic material DNA\RNA, and apoptotic macromolecules of life substance decompose and break down, and the cell debris are affinitive-absorbed into the metabolic system and excreted. Animal model tests have proven that the coating of micro-nano resin powder material on the blood vessel wall can make the fatty substances stacked on the inner wall of the blood vessel decompose and flow away with blood like a “smoke”, and make the cancerous tissues instantly breakdown into a “smoke” and flow away with blood. The volunteer is a liver cancer patient who has reached the state of clinical liver necrosis (a death notice has been received). The liver was exposed in the surgery. The micro-nano resin powder material was directly applied on the outside of the liver, such that the material rapidly penetrated into the liver tissue, and then the liver tissue began to restore blood color. In the blood drawn from the hepatic vein, cancerous cell tissue molecular debris were observed. After the liver restored blood color and the color of the drawn blood recovered normally, abdominal cavity was sutured. The whole period of the surgery was four hours. Three days later, the volunteer returned to normal diet and left hospital after one week. About a year before this case, a liver transplantation surgery was made. Because of the donor necrosis, the bad liver removed from the patient was forced to be put and immersed into the physiological saline solution of micro-nano resin powder material. Necrotic tissues made the solution turn black, while the liver in vitro restored blood color and was then implanted in the body. Without any postoperative adverse reactions, the patient soon left the hospital. In addition, the inventor and his colleagues volunteered for many trials. The effects are: oral administration can adjust the blood sugar, coating on skin can achieve whitening, eliminate subcutaneous hydrops, and quickly heal wounds and so on.

The polymer molecule of the medical absorbent resin powder material of the present invention is a chemical synthetic product, which has chemical stability, does not participate in a biochemical reaction, and has no side effect in clinical use.

Various use embodiments of the present invention are different use forms of the resin powder of the present invention, which are respectively suitable for skin coating, oral administration, injection dosage, etc.

Substantive distinguishing features of the present invention are to show the micro-nano effect and quantum effect of the medical absorbent resins.

A significant improvement provided by the present invention opened a new application field of the medical absorbent resins.

What is claimed is:

1. A micro-nano medical adsorbent resin powder material, wherein the powder material is a high-molecular-weight polymer including carbon ions, hydrogen ions, and oxygen ions; a molecular structure of the high-molecular-weight polymer includes a benzene ring and a hydroxyl group, and the high-molecular-weight polymer includes at least two or more molecules; an overall particle size of the high-molecular-weight polymer is in a range of 0.01-50 micrometer (um).

2. The micro-nano medical adsorbent resin powder material according to claim 1, wherein the high-molecular-weight polymer is a polymer including vinyl acetate and divinyl-benzene.

3. The micro-nano medical adsorbent resin powder material according to claim 1, wherein the high-molecular-weight polymer is a polymer including styrene and divinyl-benzene.

4. The micro-nano medical adsorbent resin powder material according to claim 2, wherein a monosaccharide residue or carbohydrate chains is bound to a binding site of a carbon skeleton and/or a branched-chain of the high-molecular-weight polymer to form a glycosylated polymer.

5. The micro-nano medical adsorbent resin powder material according to claim 2, wherein transition-metal ions are embedded in a carbon skeleton of the high-molecular-weight polymer.

6. The micro-nano medical adsorbent resin powder material according to claim 2, wherein transition-metal ions are embedded in a carbon skeleton of the high-molecular-weight polymer; and the metal element is iron.

7. (canceled)

8. The micro-nano medical adsorbent resin powder material according to claim 1, wherein the powder material carries a drug or a life-intervention substance, including a stem cell.

9. (canceled)

10. (canceled)

11. The micro-nano medical adsorbent resin powder material according to claim 1, wherein the powder material is added into a shaping material to make a paste, a pill, or a grease agent.

12. The micro-nano medical adsorbent resin powder material according to claim 3, wherein a monosaccharide residue or carbohydrate chains is bound to a binding site of a carbon skeleton and/or a branched-chain of the high-molecular-weight polymer to form a glycosylated polymer.

13. The micro-nano medical adsorbent resin powder material according to claim 3, wherein transition-metal ions are embedded in a carbon skeleton of the high-molecular-weight polymer.

14. The micro-nano medical adsorbent resin powder material according to claim 3, wherein transition-metal ions are embedded in a carbon skeleton of the high-molecular-weight polymer; and the metal element is iron.

15. The micro-nano medical adsorbent resin powder material according to claim 1, wherein the overall particle size is preferably 0.01-1.5 micrometer (10-1500 nm).

16. A method of applying a micro-nano medical adsorbent resin powder material, comprising:
exposing an liver by a surgeon;
applying the micro-nano resin powder material directly on an outside of the liver, and then, observing the liver restore blood color rapidly;
drawing blood from a hepatic vein;
observing a color of the blood drawn from a hepatic vein to recover normal; and then,
suturing an abdominal cavity;
wherein the micro-nano medical adsorbent resin powder material is a high-molecular-weight polymer including carbon ions, hydrogen ions, and oxygen ions; a molecular structure of the high-molecular-weight polymer includes a benzene ring and a hydroxyl group, and the high-molecular-weight polymer includes at least two or more molecules; an overall particle size is in a range of 0.01-50 micrometer (um).

17. A method of applying a micro-nano medical adsorbent resin powder material, comprising:
adding the micro-nano resin powder material into physiological saline solution;
removing a bad liver from a body of a patient;
placing the bad liver into the physiological saline solution of the micro-nano resin powder material for immerse;
observing a color of the physiological saline solution of the micro-nano resin powder material become black; and
observing the bad liver restore blood color;
replacing the bad liver into the body again; wherein the micro-nano medical adsorbent resin powder material is a high-molecular-weight polymer including carbon ions, hydrogen ions, and oxygen ions; a molecular structure of the high-molecular-weight polymer includes a benzene ring and a hydroxyl group, and the high-molecular-weight polymer includes at least two or more molecules; an overall particle size is in a range of 0.01-50 micrometer (um).
